(19) United States
(12) Patent Application Publication
(10) Pub. No.: US 2013/0102572 A1
(43) Pub. Date: Apr. 25, 2013

(54) METHODS OF TREATING SKIN CONDITIONS EXHIBITING TELANGIECTASIA

(71) Applicant: Jeffrey SUGARMAN, Santa Rosa, CA (US)

(72) Inventor: Jeffrey SUGARMAN, Santa Rosa, CA (US)

(73) Assignee: Dow Pharmaceutical Sciences, Petaluma, CA (US)

(21) Appl. No.: 13/444,576

(22) Filed: Apr. 11, 2012

Related U.S. Application Data
(60) Provisional application No. 61/474,580, filed on Apr. 12, 2011.

(51) Int. Cl. A61K 31/436; A61K 31/4164; A61K 31/194; A61K 31/65

(52) U.S. Cl. CPC A61K 31/436; A61K 31/65; A61K 31/4164; A61K 31/194
USPC 514/154; 514/291; 607/89

(57) ABSTRACT

The present application relates to methods of preventing or treating skin disorders exhibiting telangiectasia, for example, rosacea exhibiting an erythematotelangiectatic subtype. Exemplary methods comprise administering to a patient a formulation comprising a therapeutically effective amount of an mTOR inhibitor, such as rapamycin, wherein the mTOR inhibitor is the only active agent in the formulation. Suitably, the formulations are topical formulations administered to the face.

### CROSS-REFERENCE TO RELATED APPLICATIONS

The present application claims the benefit of U.S. Provisional Patent Application No. 61/474,580, filed Apr. 12, 2011, the disclosure of which is incorporated by reference herein in its entirety.

### BACKGROUND OF THE INVENTION

1. Field of the Invention

The present application relates to methods of preventing or treating skin conditions exhibiting telangiectasia, for example, rosacea exhibiting an erythematotelangiectatic subtype. Exemplary methods comprise administering to a patient a formulation comprising a therapeutically effective amount of an mTOR inhibitor, such as rapamycin, wherein the mTOR inhibitor is the only active agent in the formulation. Suitably, the formulations are topical formulations administered to the face.

2. Background of the Invention

Rapamycin (sirolimus), is a macrocyclic triene antibiotic produced by Streptomyces hygroscopicus (see U.S. Pat. No. 3,929,992) that has been shown to prevent the formation of humoral (IgE-like) antibodies in response to an albumin allergic challenge (Martel, R., Can. J. Physiol. Pharm. 55: 48 (1977)), inhibit murine T-cell activation (Staruch, M., FASEB 3: 3411 (1989)), and prolong survival time of organ grafts in histoincompatible rodents (Morris, R., Med. Sci. Res. 17: 877 (1989)). Rapamycin has been approved by the FDA to prevent rejection of organ transplants, particularly kidney transplants. It has been suggested for use in treating skin conditions, however, limited to immunoinflammatory skin diseases. See U.S. Pat. No. 5,286,730, the disclosure of which is incorporated by reference herein.

Rosacea is a chronic inflammatory condition of the facial skin affecting the blood vessels and pilosebaceous units. Rosacea exhibits four major subtypes, erythematotelangiectatic (Subtype 1), papulopustular (Subtype 2), phymatous (Subtype 3) and ocular (Subtype 4). See, e.g., Culp and Scheinfeld, “Rosacea: A Review,” P&T 43:38-45 (2009). The disease is common, especially in fair-skinned people of Celtic and northern European heritage. The onset of the disorder is usually between the ages of 30 and 50. Early stages of the disease affect women more often than men at a ratio of 3 to 1 (Jansen & Plewig, J. R. Soc. Med 90:144-150 (1997); McDonnell & Tomecki, Cleve Clin J Med. 67:587-590 (2000)). Patients typically present with Subtypes 1 and 2. Factors such as sunlight, alcohol intake, hot drinks, spicy foods, stress, and exposure to extreme heat or cold can trigger or exacerbate this disease. Rosacea may progress to the development of inflammatory lesions (papules and pustules) in association with erythema and telangiectasia. In more severe cases, patients may develop nodules and disfiguring rhinophyma. Ocular changes may occur in over half of patients.

In addition to rosacea, a number of additional skin conditions exhibit some form of telangiectasia, in which the blood vessels of the skin are abnormally or permanently dilated. Telangiectasia in combination with flushing and persistent erythema on the central face, generally referred to clinically as erythematotelangiectasia, is primarily seen in rosacea patients.

Standard treatments for rosacea include topical azelaic acid, topical metronidazole and oral tetracyclines. These therapies are generally targeted at reducing inflammatory lesions (papules, pustules) associated with rosacea, rather than the erythematotelangiectatic subtype. The most common topical therapy for rosacea is metronidazole, which is approved for the treatment of inflammatory lesions (Subtype 2) of rosacea and is available in 0.75% and 1% concentrations. FINACEA® (azelaic acid) Gel, 15% is also prescribed for treatment of papulopustular rosacea (Subtype 2). Treatment of the erythematotelangiectatic subtype of rosacea has generally been restricted to light-based treatments, including multiplexed laser. Scheinfeld, “Rosacea: A Review,” P&T 43:38-45 (2009)

A topical therapy that is effective in treating and preventing skin conditions exhibiting telangiectasia, including the erythematotelangiectatic subtype of rosacea, is desirable.

### SUMMARY OF PREFERRED EMBODIMENTS

In embodiments, methods are provided for treating a skin condition exhibiting telangiectasia comprising administering to a patient a formulation comprising a therapeutically effective amount of an mTOR inhibitor. Suitably, the mTOR inhibitor is the only active agent in the formulation. The methods suitably reduce the telangiectasia and/or erythema.

In exemplary embodiments, the mTOR inhibitor is rapamycin. Exemplary skin conditions that can be treated include, but are not limited to, rosacea, keratosis pilaris, photodamage, angiofibroma, port wine stain, cutis marmaorata telangiectatica congenita and hemangioma.

Suitably, the formulation is administered topically, for example to a patient's face. Exemplary topical formulations include, but are not limited to, a cream, a lotion, a spray, a foam, a gel, a solution, an ointment and a mask.

Suitably the mTOR inhibitor is present in the formulation at a concentration of about 0.5% to about 10% by weight, for example about 1% to about 8% by weight.

In exemplary embodiments, the formulation is administered once a day, or twice a day. The methods can also further comprise administering one or more tetracyclines orally to the patient, as well as administering vascular laser therapy to the patient.

In further embodiments, methods are provided for treating rosacea exhibiting an erythematotelangiectatic subtype. Suitable methods comprise administering topically to a patient a formulation comprising a therapeutically effective amount of rapamycin, wherein the rapamycin is the only active agent in the formulation, thereby reducing erythematotelangiectasia of the rosacea.

In additional embodiments, methods are provided for treating rosacea exhibiting an erythematotelangiectatic subtype comprising administering topically to a patient a formulation consisting essentially of a therapeutically effective amount of rapamycin, thereby reducing erythematotelangiectasia of the rosacea.

Methods are also provided for treating rosacea exhibiting only an erythematotelangiectatic subtype comprising administering topically to a patient a formulation comprising about 0.01% to about 10% rapamycin by weight, wherein rapamycin is the only active agent in the formulation, thereby reducing erythematotelangiectasia of the rosacea.

In additional embodiments, methods are provided for preventing a skin condition that exhibits telangiectasia and/or erythema comprising administering to a patient a formulation comprising a therapeutically effective amount of an mTOR inhibitor, wherein the mTOR inhibitor is the only active agent in the formulation, thereby reducing formation of the telangiectasia and/or erythema.

Additional methods are provided for preventing rosacea that exhibits an erythematotelangiectatic subtype comprising administering topically to a patient a formulation comprising a therapeutically effective amount of rapamycin, wherein said rapamycin is the only active agent in the formulation, thereby reducing formation of erythematotelangiectasia of the rosacea.

Further methods are provided for preventing rosacea that exhibits an erythematotelangiectatic subtype comprising administering topically to a patient a formulation consisting essentially of a therapeutically effective amount of rapamycin, thereby reducing formation of erythematotelangiectasia and/or erythema of the rosacea.

In additional embodiments methods are provided for preventing rosacea exhibiting only an erythematotelangiectatic subtype comprising administering topically to a patient a formulation comprising about 0.01% to about 10% rapamycin by weight, wherein the rapamycin is the only active agent in said formulation, thereby reducing formation of erythematotelangiectasia of the rosacea.

Further embodiments, features, and advantages of the embodiments, as well as the structure and operation of the various embodiments, are described in detail below.

### DETAILED DESCRIPTION OF PREFERRED EMBODIMENTS

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

In embodiments, methods are provided for preventing or treating a skin condition exhibiting telangiectasia and/or erythema. Such methods suitably comprise administering to a patient a formulation comprising a therapeutically effective amount of an mTOR inhibitor. Suitably, the mTOR inhibitor is the only active agent in the formulation. Such methods suitably reduce the telangiectasia and/or erythema exhibited by the skin condition or reduce the formation of the telangiectasia and/or erythema of the skin condition.

As used herein a “skin condition” refers to a disease, disorder or problem affecting the skin. This includes conditions that affect the face, as well as other surfaces of the skin, including back, chest, hands, neck, arms, legs, feet, trunk, etc.

The methods described herein suitably prevent a skin condition. In such preventative methods, the formulations are suitably administered prior to the development of the skin condition, so as to slow or completely stop the formation or spread of a skin condition, including the formation of the telangiectasia and/or erythema of the skin condition. Prevention of a skin condition can be utilized in patients that have previously exhibited a skin condition in the past (but no long show symptoms) or in patients that demonstrate a family history of a particular skin condition or are at an increased risk or more predisposed to develop a skin condition due to medical history and/or environmental factors.

In other embodiments, the methods described herein treat a skin condition. Treatment of a skin condition as used herein suitably includes improving a skin condition, including the telangiectasia and/or erythema exhibited by the skin condition, to a point where it is nearly or completely eliminated or cured. Treatment of a skin condition also includes the amelioration of a skin condition. To ameliorate a skin condition means to make the skin condition better, more bearable, or more satisfactory to a patient. The methods described herein can be utilized to result in any of these changes in the skin condition, or any combination thereof.

In embodiments, the methods can be used to prevent the skin condition. In further embodiments, the methods can be used to treat the skin conditions.

The skin conditions that are prevented or treated with the disclosed methods all exhibit some level of telangiectasia and/or erythema. As used herein the term “telangiectasia” or “telangiectatic” refers to abnormally and/or permanently dilated blood vessels associated with a skin condition. Telangiectasia can also be associated with increased erythema or flushing of the skin, and such a symptom is referred to as “erythematotelangiectasia” or “erythematotelangiectatic” throughout.

In embodiments, the methods comprise administering to a patient a formulation comprising a therapeutically effective amount of an mTOR inhibitor. “Patients” as used herein refer to animals, including mammals and particularly humans.

As used herein, “mTOR inhibitors” refer to inhibitors of the mammalian target of rapamycin (mTOR), which is also known as the mechanistic target of rapamycin or FK506 binding protein 12-rapamycin associated protein 1 (FRAP1). Exemplary mTOR inhibitors include, but are not limited to, rapamycin (sirolimus), temsirolimus, everolimus and ridaforolimus, etc.

Suitably, the methods comprising administering a formulation where the only active agent in the formulation is the mTOR inhibitor. That is, the formulations do not include any other additional agents that are biologically active. However, the formulations suitably comprise one or more non-active ingredients or excipients, including various emulsifiers, binders, buffers, carriers, etc., as described herein. One or more of these non-active ingredients may have beneficial effects on the skin, for example adding or removing moisture or oil from the skin, lubricating the skin, etc., but they are not considered active agents.

In additional embodiments, the methods can comprise administering a formulation comprising an mTOR inhibitor and one more additional active agent. Exemplary active agents that can be used in the formulations are known in the art and include, for example, various antibiotics, steroids, etc.

Suitably, the methods described herein result in reduction of the telangiectasia and/or erythema exhibited by the skin condition. That is, the dilation or abnormal configuration of the blood vessels of the skin condition, and/or the redness or flushing, is less than that relative to the dilation or abnormal configuration of the blood vessels, and/or the redness or flushing, of the skin condition prior to administering the mTOR inhibitor. Or the dilation or abnormal configuration of the blood vessels of the skin condition, and/or the redness or flushing, is less than the dilation or abnormal configuration of the blood vessels, and/or the redness or flushing, of the same skin condition that is not being administered the mTOR inhibitor. Thus, the reduction of the telangiectasia and/or erythema can be determined by comparing the patient's skin condition prior to the beginning of the administration to a time after the administration started, or the reduction can be determined by comparing the patient's skin condition after beginning the administration relative to a patient that also has the skin condition, but is not receiving the mTOR inhibitor.

In embodiments in which the methods are used to prevent the skin condition, the reduction in formation of telangiectasia and/or erythema is suitably determined by comparing the patient's skin condition after beginning the administration relative to a patient that has the same skin condition, but is not receiving the mTOR inhibitor.

Reduction of the telangiectasia and/or erythema can be determined via visual examination, including both human clinical evaluation and the use instrumentation (including photography and image analysis software) to visualize the change in the telangiectasia and/or erythema. The reduction can also be determined via measurement of the blood flow or other characteristics of the skin and blood vessels that can be used to determine whether or not the telangiectasia and/or erythema has been reduced. Examples of instrumentation that can be utilized to determine the change in telangiectasia and/or erythema include, but are not limited to, a Chroma Meter (KONICA MINOLTA®, Ramsey, N.J.), laser Doppler blood flow meters, etc.

Suitably, the evaluation of the skin condition and specifically the level of telangiectasia and/or erythema will be determined by a trained medical professional evaluating the patient utilizing an investigative global assessment of the skin condition. Typically, such global assessments assign a value to the degree of telangiectasia and/or erythema exhibited by the skin condition. This investigative global assessment value can range, for example, from 0 (Absent) to 1 (Mild) to 2 (Moderate) to 3 (Significant) to 4 (Severe), including values between these numeric gradings (e.g., intervals of 0.1). In addition to the assessment made by the medical professional, the patient's input and observations of their skin condition and responses to various inquiries (e.g., stinging or burning sensations) also play a role in determining the investigative global assessment value that is assigned.

Suitably, the telangiectasia and/or erythema will be reduced, either relative to the level of telangiectasia and/or erythema prior to starting the administration, or as compared to a patient that is not receiving the mTOR inhibitor. That is, the investigative global assessment assigned to the telangiectasia and/or erythema/skin condition after administration will be less than the investigative global assessment assigned to the telangiectasia and/or erythema/skin condition prior to the start of administration. In the case of prevention, the investigative global assessment will be less than the investigative global assessment as compared to a patient that is not receiving the mTOR inhibitor.

The reduction can take place over a period of days to weeks to months to years. For example, a patient may receive an investigative global assessment assigned to the telangiectasia and/or erythema/skin condition of 3.5 prior to the beginning of administration, and an investigative global assessment assigned to the telangiectasia and/or erythema/skin condition of 3.0 after two weeks of administration in accordance with the methods described herein. It should be noted that any reduction in the numerical value provided by the investigative global assessment is considered a reduction in the telangiectasia and/or erythema exhibited by the skin condition. For example, a reduction in the assigned value of about 4.0 or less, a reduction of about 3.5 or less, a reduction of about 10 or less, a reduction of about 2.5 or less, a reduction of about 2.0 or less, a reduction of about 1.5 or less, a reduction of about 1.0 or less, or a reduction of about 0.5 or less, is considered a reduction in the telangiectasia and/or erythema exhibited by the skin condition.

In embodiments where instrumentation (including photography and image analysis software) is utilized in the determination of the reduction in the telangiectasia and/or erythema exhibited by the skin condition, the reduction can be illustrated by a percent (%) reduction. For example, reduction of the telangiectasia and/or erythema includes reduction in the measurement made by the instrumentation by at least about 10%, suitably, at least about 20%, at least about 30%, at least about 40%, at least about 50%, at least about 60%, at least about 70%, at least about 80%, at least about 90% or at least about 95%. In some embodiments, the telangiectasia and/or erythema will be completely eliminated, resulting in a reduction of about 100% of the measurement made by the instrumentation.

In embodiments where the methods are used for prevention of a skin condition, the reduction in formation of the telangiectasia and/or erythema can take place over a period of days to weeks to months to years. For example, a patient receiving the preventative administration may receive an investigative global assessment assigned to the telangiectasia and/or erythema/skin condition of 2.0, whereas a patient that is exhibiting the telangiectasia and/or erythema/skin condition, but is not receiving the preventative administration, may receive an investigative global assessment assigned to the telangiectasia and/or erythema/skin condition of 3.0 after two weeks of administration in accordance with the methods described herein. It should be noted that any reduction in the numerical value provided by the investigative global assessment is considered a reduction in the formation of the telangiectasia and/or erythema exhibited by the skin condition. For example, a reduction in the assigned value of about 4.0 or less, a reduction of about 3.5 or less, a reduction of about 3.0 or less, a reduction of about 2.5 or less, a reduction of about 2.0 or less, a reduction of about 1.5 or less, a reduction of about 1.0 or less, or a reduction of about 0.5 or less, is considered a reduction in the formation of the telangiectasia and/or erythema exhibited by the skin condition.

In embodiments where instrumentation (including photography and image analysis software) is utilized in the determination of the reduction in the formation of the telangiectasia and/or erythema exhibited by the skin condition, the reduction can be illustrated by a percent (%) reduction relative to a patient that is not receiving the preventative administration. For example, reduction of the telangiectasia and/or erythema includes reduction in the measurement made by the instrumentation by at least about 10%, suitably, at least about 20%, at least about 30%, at least about 40%, at least about 50%, at least about 60%, at least about 70%, at least about 80%, at least about 90% or at least about 95%. In some embodiments, the telangiectasia and/or erythema will be completely eliminated, resulting in a reduction of about 100% of the measurement made by the instrumentation.

Exemplary skin conditions that can be prevented or treated utilizing the methods described herein include, but are not limited to, rosacea, keratosis pilaris, photodamage, angiofibroma, port wine stain, cutis marmaorata telangiectatica congenital, hemangioma, scleroderma, hereditary hemorrhagic telangiectasia (Olser-Rendu syndrome), Ataxia-Telangiectasia, spider angioma, Bloom syndrome, Klippel-Trenaunay-Weber syndrome, Sturge-Weber disease, Xeroderma pigmentosa and Nevus flammeus.

As described herein, suitably the mTOR inhibitor that is administered to the patient is rapamycin. Administration of the mTOR inhibitor can be via any suitable route, including for example, orally, intravenously, subdermally, peritoneally, via inhalation, intramuscularly, intradermally, intraocularly, intranasally or subcutaneously.

Suitably, the mTOR inhibitor is formulated for topical administration and administered topically to the surface of the skin where the skin condition is present. In embodiments, the formulations are administered topically to a patient's face, which includes the central region of the face, as well as the forehead, nose, ears and cheek/mouth areas.

Exemplary topical formulations for use in the methods described herein include, but are not limited to, a cream, a lotion, a spray, a gel, a solution, a foam, a suspension, a patch an ointment and a mask, as well as other similar topical formulations known in the art.

Exemplary non-active ingredients or excipients for use in the topical formulations described herein are well known in the art. Such non-active ingredients include, but are not limited to, humectants, emollients, pH stabilizing agents, preservatives, chelating agents, gelling agents, thickening agents, emulsifiers, binders, buffers, carriers, anti-oxidants, etc., as described, for example, in Remington: The Science and Practice of Pharmacy, 21^{st} Edition, Lippincott Williams & Wilkins, Philadelphia, Pa. (2006); see also, U.S. Food & Drug Administration, Inactive Ingredients for Approved Drugs, available online.

In exemplary embodiments, the mTOR inhibitor, suitably rapamycin, is present in the formulations at a concentration of about 0.01% to about 20% by weight, about 0.05% to about 20% by weight, about 0.1% to about 20% by weight, more suitably about 0.2% to about 18%, about 0.4% to about 16%, 0.5% to about 15% by weight, or about 0.5% to about 10% by weight, about 1% to about 8% by weight, or about 1%, about 2%, about 3%, about 4%, about 5%, about 6%, about 7% or about 8% by weight, and any values between these values.

Amounts of non-active agents, including those disclosed herein, are readily determinable by those of ordinary skill in the art. Generally, the amounts and ratios of the non-active agents are determined so as to provide the topical formulations with the desired consistency, stability, delivery characteristics and other properties of the formulation.

For example, suitable topical formulations comprising an mTOR inhibitor, such as rapamycin, can include the following non-active ingredients in the ranges indicated. All percentages provided are weight percentages.

Exemplary solution or spray: about 1% to about 8% mTOR inhibitor (rapamycin), about 20% to about 50% dimethyl sulfoxide (DMSO), about 10% to about 20% propylene glycol, about 10% to about 40% poly(ethylene glycol) 400 (400 molecular weight (MW)) (PEG 400), about 5% to about 10% Transcutol and about 0% to about 50% water.

Exemplary gel: about 1% to about 8% mTOR inhibitor (rapamycin), about 20% to about 50% DMSO, about 10% to about 20% PG, about 10% to about 40% PEG 400, about 5% to about 10% Transcutol, about 1% to about 2% Gelling Agents, and about 0% to about 50% Water.

Exemplary cream or lotion: about 1% to about 8% mTOR inhibitor (rapamycin), about 1% to about 5% mineral oil, about 0.5% to about 2% stearyl alcohol, about 0.5% to about 2% acetyl alcohol, about 1% to about 3% Brij 21, about 1% to about 3% Brij 721, about 0% to about 15% emulsifying wax, about 0% to about 5% glycerol monostearate (GMS), about 0% to about 5% Isopropyl Myristate (IPM), about 0.25% to about 1% Carbopol and about 60% to about 80% Water.

Exemplary foam: about 1% to about 8% mTOR inhibitor (rapamycin), about 5% to about 10% PG, about 10% to about 50% DMSO, about 10% to about 40% PEG 400, about 0% to about 50% Water and propellants.

Exemplary ointment: about 1% to about 8% mTOR inhibitor (rapamycin), about 10% to about 40% PEG 400, about 10% to about 30% PEG 3350 (3350 MW PEG), about 5% to about 10% PG and about 0% to about 70% Petrolatum.

The topical formulations can also comprise suitable preservatives, including for example, about 0% to about 0.2% Methylparaben, about 0% to about 0.03% Propylparaben or about 0% to about 5% Benzyl Alcohol.

Suitable dosing regimens for administering the formulations described herein can be readily determined by those of ordinary skill in the art. In exemplary embodiments, the formulations are administered to the patient once a day, twice a day or three times a day, etc. The duration of the administration is dependent on the skin condition, the severity of the skin condition, and the patient's response to the formulation. Suitably the duration of administration can be from about a few days to several weeks. In other embodiments, the administration can be for a period of about a few days to several weeks, and then discontinued until it is desirable or necessary to administer the formulations again. For example, the formulations can be administered for an initial period to reduce the telangiectasia and/or erythema, and then stopped until it is desirable or necessary to reduce the telangiectasia and/or erythema at a later time.

In embodiments, the methods described herein can further comprise administering one or more tetracyclines or other antibiotics orally to the patient. The dosage and timing of the administration of tetracyclines or other antibiotics can be readily determined by those of ordinary skill in the art. The methods can also further comprise administering metronidazole or azelic acid topically (e.g., to the face) of the patient. Suitable formulations of metronidazole are available in 0.75% and 1% concentrations, and FINACEA® (azelaic acid) Gel, 15%, is also available.

In additional embodiments, the methods can further comprise administering vascular laser therapy to the patient. Techniques and methods for administering vascular laser therapy (i.e., multiplexed pulsed dye laser therapy) to a patient are well known in the art, for example, as disclosed in U.S. Pat. No. 6,306,130; and Larson et al., “Recalcitrant rosacea successfully treated with multiplexed pulsed dye laser,” J. Drugs Dermatol. 6:843-845 (2007), the disclosures of each of which are incorporated by reference herein in their entireties. The vascular laser therapy can be administered at the same time as the patient is receiving the mTOR inhibitor, or the vascular laser therapy can be given after the end of the mTOR administration.

In further embodiments, methods are provided for preventing or treating rosacea exhibiting an erythematotelangiectatic subtype. Such methods suitably comprise administering topically to a patient a formulation comprising a therapeutically effective amount of rapamycin, wherein the rapamycin is the only active agent in the formulation. The methods suitably reduce erythematotelangiectasia of the rosacea or reduce the formation of the erythematotelangiectasia.

As used herein the term “rosacea” includes and is a synonym for acne rosacea, and refers to a chronic inflammatory condition of the facial skin affecting the blood vessels and pilosebaceous units. Rosacea exhibits four major subtypes, erythematotelangiectatic, papulopustular, phymatous and ocular. See, e.g., Culp and Scheinfeld, “Rosacea: A Review,” P&T 43:38-45 (2009).

The methods described herein are suitably used for prevention or treatment of rosacea that exhibits an erythematotelangiectatic subtype. As used herein “erythematotelangiectatic” refers to rosacea that exhibits flushing and persistent central facial erythema with the presence of telangiectases (abnormal or dilated blood vessels). In exemplary embodiments, the rosacea exhibits erythematotelangiectatic along with conditions of the other of the subtypes. In other embodiments, only an erythematotelangiectatic subtype is exhibited by the rosacea. That is, no characteristics of the other four subtypes are present in the rosacea.

As described herein, suitably the formulations are topical formulations, including a cream, a lotion, a spray, a gel, a solution, a suspension, a foam, a patch, an ointment and a mask, and are administered to the patient's face.

In exemplary embodiments, rapamycin, is present in the topical formulations at a concentration of about 0.1% to about 20% by weight, more suitably about 0.2% to about 18%, about 0.4% to about 16%, 0.5% to about 15% by weight, or about 0.5% to about 10% by weight, about 1% to about 8% by weight, or about 1%, about 2%, about 3%, about 4%, about 5%, about 6%, about 7% or about 8% by weight.

Suitably, the methods described herein result in reduction of the erythematotelangiectasia exhibited by the rosacea. That is, the flushing and persistent central facial erythema with the presence of dilation or abnormal configuration of the blood vessels of the rosacea is less than that relative to the flushing and persistent central facial erythema with the presence of dilation or abnormal configuration of the blood vessels of the rosacea prior to administering rapamycin. Or the flushing and persistent central facial erythema with the presence of dilation or abnormal configuration of the blood vessels of the rosacea is less than the flushing and persistent central facial erythema with the presence of dilation or abnormal configuration of the blood vessels of rosacea that is not being administered the rapamycin. Thus, the reduction of the erythematotelangiectasia can be determined by comparing the patient's rosacea prior to the beginning of the administration of the rapamycin to a time after the administration, or the reduction can be determined by comparing the patient's rosacea after beginning the administration of the rapamycin relative to a patient that also has rosacea, but is not receiving the rapamycin.

In embodiments in which the methods are used to prevent rosacea, the reduction information of the rosacea is suitably determined by comparing the patient's skin condition after beginning the administration relative to a patient that has the skin condition, but is not receiving the mTOR inhibitor.

Reduction of the erythematotelangiectasia, or reduction in the formation of the erythematotelangiectasia, can be determined via visual examination (including photography and image analysis software), including both human clinical evaluation and the use of instrumentation to visualize the change in the erythematotelangiectasia. The reduction can also be determined via measurement of the blood flow or other characteristics of the skin and blood vessels that can be used to determine whether or not the erythematotelangiectasia has been reduced. Examples of instrumentation that can be utilized to determine the change in erythematotelangiectasia include, but are not limited to, a Chroma Meter (KONICA MINOLTA®, Ramsey, N.J.), laser Doppler blood flow meters, etc.

Suitably, the evaluation of the rosacea and specifically the level of erythematotelangiectasia will be determined by a trained medical professional evaluating the patient utilizing an investigative global assessment of the rosacea. Typically, such global assessments assign a value to the degree of erythematotelangiectasia exhibited by the rosacea. This investigative global assessment value can range, for example, from 0 (Absent) to 1 (Mild) to 2 (Moderate) to 3 (Significant) to 4 (Severe), including values between these numeric gradings (e.g., intervals of 0.1). In addition to the assessment made by the medical professional, the patient's input and observations of their condition and responses to various inquiries (e.g., stinging or burning sensations) also play a role in determining the investigative global assessment value to assign.

Suitably, the erythematotelangiectasia will be reduced, either relative to the level of erythematotelangiectasia prior to starting the administration, or as compared to a patient that is not receiving the formulation. That is, the investigative global assessment assigned to the erythematotelangiectasia/rosacea after administration will be less than the investigative global assessment assigned to the erythematotelangiectasia/rosacea prior to the start of administration. In the case of prevention, the investigative global assessment will be less than the investigative global assessment as compared to a patient that is not receiving the rapamycin.

The reduction can take place over a period of days to weeks to months. For example, a patient may receive an investigative global assessment assigned to the erythematotelangiectasia/rosacea of 3.5 prior to the beginning of administration, and an investigative global assessment assigned to the erythematotelangiectasia/rosacea of 3.0 after two weeks of administration in accordance with the methods described herein. It should be noted that any reduction in the numerical value provided by the investigative global assessment is considered a reduction in the erythematotelangiectasia exhibited by the rosacea. For example, a reduction in the value of about 4.0 or less, a reduction of about 3.5 or less, a reduction of about 3.0 or less, a reduction of about 2.5 or less, a reduction of about 2.0 or less, a reduction of about 1.5 or less, a reduction of about 1.0 or less, or a reduction of about 0.5 or less, is considered a reduction in the erythematotelangiectasia exhibited by the rosacea. This reduction in numerical value can also be represented as a percentage reduction, based on the initial (baseline) value assessed.

In embodiments where instrumentation (including photography and image analysis software) is utilized in the determination of the reduction in the erythematotelangiectasia exhibited by the rosacea, the reduction can also be illustrated by a % reduction. For example, reduction of the erythematotelangiectasia includes reduction in the measurement made by the instrumentation by at least about 10%, suitably, at least about 20%, at least about 30%, at least about 40%, at least about 50%, at least about 60%, at least about 70%, at least about 80%, at least about 90% or at least about 95%. In some embodiments, the erythematotelangiectasia will be completely eliminated, resulting in a reduction of about 100% of the measurement made by the instrumentation.

Suitable dosing regimens for administering the rapamycin-comprising formulations described herein can be readily determined by those of ordinary skill in the art. In exemplary embodiments, the formulations are administered to the patient once a day, twice a day or three times a day, etc. The duration of the administration is dependent upon the severity of the rosacea, and the patient response to the formulation, and suitably can be from about a few days to several weeks to months if desired. In other embodiments, the administration can be used for a period of about a few days to several weeks to months if desired, and then discontinued until it is desirable necessary to administer the formulations again. For example, the formulations can be administered for an initial period to reduce the erythematotelangiectasia, and then stopped until it is desirable or necessary to reduce the erythematotelangiectasia at a later time.

In embodiments, the methods described herein can further comprise administering one or more tetracyclines or other antibiotics orally to the patient. The dosage and timing of the administration of tetracyclines or other antibiotics can be readily determined by those of ordinary skill in the art. The methods can also further comprise administering metronidazole or azelic acid topically (e.g., to the face) of the patient. Suitable formulations of metronidazole are available in 0.75% and 1% concentrations, and FINACEA® (azelaic acid) Gel, 15%, is also available. In additional embodiments, the methods can further comprise administering vascular laser therapy to the patient.

As described herein, in embodiments, the formulations administered to the patients specifically exclude active agents other than rapamycin. Thus, in suitable embodiments, methods of preventing or treating rosacea exhibiting an erythematotelangiectatic subtype are provided. Such methods suitably comprise administering topically to a patient a formulation consisting essentially of a therapeutically effective amount of an mTOR inhibitor, such as rapamycin. The methods suitably result in the reduction of erythematotelangiectasia of the rosacea or reduction in the formation of the erythematotelangiectasia.

The addition of active agents other than mTOR inhibitors to the formulations, for example active agents other than rapamycin, is considered a material alteration to such formulations and is thus excluded from such formulations that consist essentially of mTOR inhibitors or that consist essentially of rapamycin.

As described throughout, suitable formulations for use in the practice of the methods described herein are topical formulations that are administered to the surface of the skin, including a patient's face. Exemplary topical formulations include, but are not limited to, a cream, a foam, a lotion, a spray, a gel, a solution, an ointment and a mask.

In the disclosed topical formulations that consist essentially of an mTOR inhibitor, or consist essentially of rapamycin, other active agents are specifically excluded. However, such topical formulations can comprise other non-active agents, including for example, humectants, emollients, pH stabilizing agents, preservatives, chelating agents, gelling agents, thickening agents, emulsifiers, binders, buffers, carriers, anti-oxidants, etc. Examples of suitable types of non-active agents and amounts of such agents that can be included in the topical formulations are described throughout.

In exemplary embodiments, the formulations that consist essentially of an mTOR inhibitor, suitably rapamycin, the rapamycin is present in at a concentration of about 0.1% to about 20% by weight, more suitably about 0.2% to about 18%, about 0.4% to about 16%, 0.5% to about 15% by weight, or about 0.5% to about 10% by weight, about 1% to about 8% by weight, or about 1%, about 2%, about 3%, about 4%, about 5%, about 6%, about 7% or about 8% by weight.

In exemplary embodiments, the formulations that consist essentially of rapamycin are administered to the patient once a day, twice a day or three times a day, etc. The duration of the administration is dependent on the skin condition, the severity of the skin condition, and the patient response to the formulation, and suitably can be from about a few days to several weeks. In other embodiments, the administration can be used for a period of about a few days to several weeks, and then discontinued until it is desirable or necessary to administer the formulations again. For example, the formulations can be administered for an initial period to reduce the telangiectasia, and then stopped until it is desirable or necessary to reduce the telangiectasia at a later time.

In embodiments, the methods described herein can further comprise administering one or more tetracyclines or other antibiotics orally to the patient. The dosage and timing of the administration of tetracyclines or other antibiotics can be readily determined by those of ordinary skill in the art. The methods can also further comprise administering metronidazole or azelic acid topically (e.g., to the face) of the patient. Suitable formulations of metronidazole are available in 0.75% and 1% concentrations, and FINACEA® (azelaic acid) Gel, 15%, is also available. In additional embodiments, the methods can further comprise administering vascular laser therapy to the patient.

In additional embodiments, methods for preventing or treating rosacea exhibiting only an erythematotelangiectatic subtype are provided. Suitably such methods comprise administering topically to a patient a formulation comprising about 0.5% to about 10% rapamycin by weight, wherein said rapamycin is the only active agent in the formulation. The methods suitably reduce erythematotelangiectasia of the rosacea or reduce the formation of the erythematotelangiectasia.

Exemplary topical formulations, including additional non-active agents, are described throughout. Dosing regimens and schedules for preventing or treating rosacea exhibiting only an erythematotelangiectatic subtype are described throughout, as are suitably amounts of rapamycin that can be included in the formulations.

It will be readily apparent to one of ordinary skill in the relevant arts that other suitable modifications and adaptations to the methods and applications described herein can be made without departing from the scope of any of the embodiments. The following examples are included herewith for purposes of illustration only and are not intended to be limiting.

### EXAMPLES

### Example 1

### Administration of Topical Formulation Comprising Rapamycin for Treatment of Rosacea

### Formulation

Topical formulations of rapamycin are prepared in a suitable cream or lotion formulation. The amount of rapamycin is varied in the formulations from about 1% to about 8% by weight.

### Administration to Patients

Patients diagnosed with rosacea exhibiting an erythematotelangiectatic subtype are selected for administration of the topical formulations. The topical formulations are applied to the skin surface of the patients' faces once a day or twice a day.

The patients are monitored once a week for signs of reduced erythematotelangiectasia of the rosacea. The monitoring includes physical clinical examination and assignment of an investigative global assessment by a trained medical professional, as well as instrumental monitoring and imaging of the rosacea.

Changes in the erythematotelangiectasia of the rosacea are monitored over the administration period and recorded relative to the erythematotelangiectasia of the rosacea at the start of the administration. Changes in the erythematotelangiectasia of the rosacea are also recorded relative to a similar patient that is not administered rapamycin.

### Example 2

### Administration of Topical Formulation Comprising Rapamycin for Prevention of Rosacea

### Formulation

Topical formulations of rapamycin are prepared in a suitable cream or lotion formulation. The amount of rapamycin is varied in the formulations from about 1% to about 8% by weight.

### Administration to Patients

Patients previously diagnosed with rosacea that exhibited an erythematotelangiectatic subtype, but that are not currently presenting with rosacea, are selected for administration of the topical formulations, in one population.

Patients with an enhanced or increased risk of developing rosacea, but that are not currently presenting with rosacea, are also selected for administration of the topical formulations, in a second population.

The topical formulations are applied to the skin surface of the patient's faces once a day or twice a day.

The patients are monitored once a week or signs of the presence of erythematotelangiectasia of the rosacea. The monitoring includes physical clinical examination and assignment of an investigative global assessment by a trained medical professional, as well as instrumental monitoring and imaging of the rosacea.

Development of any erythematotelangiectasia of the rosacea is monitored over the administration period and recorded relative to the patient's skin at the time of starting the administration. Development of any erythematotelangiectasia of the rosacea is also recorded relative to a similar patient that is not administered rapamycin, but that is now developing rosacea.

### Example 3

### Administration of Topical Formulation Comprising Rapamycin for Treatment of Rosacea

The purpose of this study is to evaluate the efficacy of rapamycin for the treatment of erythematotelangiectatic rosacea (ETR) (Subtype 1). The Subtype 1 of rosacea is characterized by flushing and persistent central facial erythema. There is often accompanying telangiectasia. There may be associated stinging, burning, roughness, and scaling. The National Rosacea Society estimates that 40% of America's 14 million rosacea sufferers experience Subtype 1 ETR. In addition, 60% of physicians surveyed indicate that reduction/treatment of erythema is the greatest unmet need in rosacea treatment.

### Formulation

Topical Rapamycin Ointment 1%: 40 mg of rapamycin was obtained using 40 tablets of RAPAMUNE® (Wyeth-Ayerst) containing 1 mg of rapamycin each. The tablets were crushed using a mortar and pestle and then sifted to remove the colored pieces of tablet coating material. The rapamycin was next extracted from the crushed tablets with three sequential aliquots of acetone by shaking well in a tightly closed glass jar. The aliquots were passed through a filter paper to exclude the tablet excipients, and the filtrate was air dried for at least 8 hours on a glass plate. The resulting pure rapamycin was then incorporated into 4 grams of petrolatum and mixed until homogeneous.

### Study Procedure

An open label, single center study was performed in 1 subject with moderate ETR (defined as an Investigator Global Assessment (IGA) score of 3 on a scale of 0-4). The subject applied study drug to the right cheek once a day (in the morning) for a 2-week period.

Efficacy assessments were based on the Evaluator's evaluations of the signs and symptoms of ETR. At Baseline and at Week 2 the subject was evaluated by the Evaluator as per the IGA of ETR.

Safety was assessed by the occurrence of adverse events (AEs) and localized cutaneous tolerability (burning, stinging, dryness, scaling, and itching).

### Investigator Global Assessment (IGA)

The Investigator Global Assessment (IGA) for rosacea is a 5-point, static global assessment ranging from clear to severe that is independent of the Baseline score. The Evaluator makes the assessment without referring to the Baseline value or any previous study visits.

At all visits for the subject, the IGA is determined by the Evaluator according to the scale below. To qualify for the study, the subject must have a score of 3 or 4 (moderate or severe) rosacea at Baseline. Table 1 provides the definitions and guidelines.

TABLE 1
IGA for Erythematotelangiectatic Rosacea

| Score | Grade | Definition |
|---|---|---|
| 0 | Clear | No erythema; |
| | | No or very mild telangiectasia |
| 1 | Almost Clear | Mild, transient erythema may be present; |
| | | Mild telangiectasia may be present |
| 2 | Mild | Mild, non-transient erythema may be present |
| | | Mild telangiectasia may be present |
| 3 | Moderate | Moderate non-transient erythema may be present with |
| | | mild flushing; |
| | | Moderate telangiectasia is present |
| 4 | Severe | Severe persistent non-transient erythema may be |
| | | present with severe flushing |
| | | Severe telangiectasia may be present |

### Cutaneous Tolerability Evaluation

Cutaneous tolerability is evaluated by assessing the signs and symptoms of scaling, dryness, itching, burning, and stinging according to the following static (without reference to any prior visits) scales and definitions at Baseline and each follow up visit.

Scaling:

0—None No scaling

1—Mild Barely perceptible, fine scales/limited areas of the face

2—Moderate Fine scale generalized to all areas of the face

3—Severe Scaling and peeling of skin over all areas of the face

Dryness:

0—None No dryness

1—Mild Slight, but definite dryness

2—Moderate Definite dryness

3—Severe Marked dryness

Itching:

0—None No itching

1—Mild slight itching, not really bothersome

2—Moderate Definite itching that is somewhat bothersome

3—Severe Intense itching that may interrupt daily activities and/or sleep

Burning:

0—None No burning

1—Mild Slight burning sensation; not really bothersome

2—Moderate Definite warm, burning that is somewhat bothersome

3—Severe Hot burning sensation that causes definite discomfort and may interrupt daily activities and/or sleep

Stinging:

0—None No stinging

1—Mild Slight stinging sensation, not really bothersome

2—Moderate Definite stinging sensation that is somewhat bothersome

3—Severe Stinging sensation that causes definite discomfort and may interrupt daily activities and/or sleep

The subject was not allowed to use on the face of over-the-counter products or cosmetics that contain benzoyl peroxide, alpha-hydroxy acid, salicylic acid, retinol, glycolic acid, or sunscreen, nor to have any cosmetic procedures (e.g. superficial chemical peels, exfoliation or microdermabrasion of the face) within the past 2 months. The subject could not use of any topical or oral prescription for rosacea on the face within 4 weeks prior to the Baseline visit, could not use of laser or light based rosacea treatments in the past 2 months. The subject had to undergo the specified washout period(s) for the following topical preparations:

Topical astringents and abrasives 1 week

Moisturizers* or sunscreens on the face 1 week

Antibiotics** on the face 2 weeks

Other topical anti-rosacea drugs, e.g., sodium sulfacetamide, azelaic acid 1 week

Soaps containing antimicrobials 1 week

Anti-inflammatory drugs, such as corticosteroids, calcineurin inhibitors on the face* 2 weeks

Prescription Retinoids, including retinol 4 weeks

The subject had to undergo the specified washout period(s) for any of the following oral preparations:

Nonsteroidal anti-inflammatory drugs (NSAIDs)***: 2 weeks

Corticosteroids (oral and parenteral)̂: 4 weeks

Antibiotics (oral)**: 4 weeks

Other systemic rosacea treatments: 4 weeks

Vasodilators or alpha-adrenergic receptor blocking agents: 4 weeks

Laser or light based treatment on face: 2 months

Systemic retinoids: 6 months

* The use of a facial moisturizer and/or make up regimen (as applicable) must be stable for 2 months preceding study enrollment and not expected to change during the course of the study. Applications of the moisturizer and/or makeup are not allowed until after the study assessments are completed on the days of the clinic visits.

** Antibiotic treatment of an infection is allowed during the study if use is limited to ≦2 weeks in total duration.

*** NSAIDs used during the study must be limited to ≦2 weeks in total duration. A stable regimen of low dose aspirin in the 2 months preceding study enrollment (e.g. for preventive therapy in subject with coronary artery disease) is allowed if the treatment regimen is not expected to change during the course of the study.

̂ a stable regimen of inhaled corticosteroids or intra-articular corticosteroids for stable medical conditions in the 2 months preceding study enrollment are allowed

### Baseline Visit (Day 0)

1. The Evaluator performed the IGA for ETR which was 3 (moderate)

2. Confirmed that the subject meets all study inclusion/exclusion criteria

3. The study technician explained the proper technique for application of the study drug for subject(s) enrolled

4. A photo of the face was taken

5. The technician weighed and dispensed 1 tube of study drug to the subject

6. The Investigator/Evaluator completed the Cutaneous Tolerability Evaluations which revealed that the subject did not have any stinging, burning, dryness or itching in the affected areas.

### Follow up Visit (Day 14)

1. The subject did not have any concomitant medications since the previous visit.

2. There were not changes to other skin care products (moisturizers, sunscreens, soaps, etc.) on the other skin care source.

3. There were no new adverse events reported spontaneously by the subject or changes in any ongoing adverse events.

4. Evaluator performed the IGA for ETR which revealed an IGA of 2 (mild). This represents an improvement of the IGA from a 3 (moderate) to a 2 (mild) in just 2 weeks

5. Photograph(s) of the face were taken.

6. The Evaluator completed the Cutaneous Tolerability Evaluations which revealed that the subject did not have any stinging, burning, dryness or itching in the affected areas.

7. The study technician weighed the study drug tube.

The subject is to be evaluated at specified periods over the course of 1, 3, 6, 9, 12, 24, and 48/or months, etc. An improvement from the IGA of 3 (moderate) to 2 (mild) to 1 (almost clear) to 0 (clear) over the study period, and potentially maintaining this improvement, is an indication of the surprising and unexpected reduction in IGA demonstrated by the present formulations.

Quantitative evaluation of the subject's skin, including Chroma Meter analysis, laser Doppler blood flow meter analysis and/or image analysis of the photographs of the subject, are also to be made, confirming the improvement in the clinical condition of the subject. These improvements can be shown by a reduction or change in the measurement made by the instrumentation/image analysis by at least about 10%, suitably, at least about 20%, at least about 30%, at least about 40%, at least about 50%, at least about 60%, at least about 70%, at least about 80%, at least about 90% or at least about 95%. This change can be measured as either the change versus the initial or baseline reading, or versus an untreated area of the face, representing a control. In some embodiments, the erythematotelangiectasia will be completely eliminated, resulting in a reduction or change of about 100% of the measurement made by the instrumentation.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

What is claimed is:

1. A method for treating a skin condition exhibiting telangiectasia comprising administering to a patient a formulation comprising a therapeutically effective amount of an mTOR inhibitor, wherein said mTOR inhibitor is the only active agent in said formulation, thereby reducing said telangiectasia.

2. The method of claim 1, wherein said mTOR inhibitor is rapamycin.

3. The method of claim 1, wherein said skin condition is selected from the group consisting of rosacea, keratosis pilaris, photodamage, angiofibroma, port wine stain, cutis marmaorata telangiectatica congenita and hemangioma.

4. The method of claim 1, wherein said formulation is administered topically.

5. The method of claim 4, wherein said formulation is administered to said patient's face.

6. The method of claim 4, wherein said formulation is selected from the group consisting of a cream, a lotion, a spray, a foam, a gel, a solution, a suspension, a patch, an ointment and a mask.

7. The method of claim 1, wherein said mTOR inhibitor is present in said formulation at a concentration of about 0.01% to about 10% by weight.

8. The method of claim 7, wherein said mTOR inhibitor is present in said formulation at a concentration of about 1% to about 8% by weight.

9. The method of claim 1, wherein said formulation is administered once a day.

10. The method of claim 1, wherein said formulation is administered twice a day.

11. The method of claim 1, further comprising administering one or more tetracyclines orally to said patient.

12. The method of claim 1, further comprising administering metronidazole or azelic acid topically to said patient.

13. The method of claim 1, further comprising administering vascular laser therapy to said patient.

14. A method for treating rosacea exhibiting an erythematotelangiectatic subtype comprising administering topically to a patient a formulation comprising a therapeutically effective amount of rapamycin, wherein said rapamycin is the only active agent in said formulation, thereby reducing erythematotelangiectasia of said rosacea.

15. The method of claim 14, wherein said formulation is administered to said patient s face.

16. The method of claim 14, wherein said formulation is selected from the group consisting of a cream, a lotion, a spray, a foam, a gel, a solution, a suspension, a patch, an ointment and a mask.

17. The method of claim 14, wherein said rapamycin is present in said formulation at a concentration of about 0.01% to about 10% by weight.

18. The method of claim 17, wherein said rapainycin is present in said formulation at a concentration of about 1% to about 8% by weight.

19. The method of claim 14, wherein said rosacea exhibits only an erythematotelangiectatic subtype.

20. The method of claim 14, wherein said formulation is administered once a day.

21. The method of claim 14, wherein said formulation is administered twice a day.

22. The method of claim 14, further comprising administering one or more tetracyclines orally to said patient.

23. The method of claim 14, further comprising administering metronidazole or azelic acid topically to said patient.

24. The method of claim 14, further comprising administering vascular laser therapy to said patient.

25. A method for treating rosacea exhibiting an erythematotelangiectatic subtype comprising administering topically to a patient a formulation consisting essentially of a therapeutically effective amount of rapamycin, thereby reducing erythematotelangiectasia of said rosacea.

26. The method of claim 25, wherein said formulation is administered to said patient's face.

27. The method of claim 25, wherein said formulation is selected from the group consisting of a cream, a lotion, a spray, a foam, a gel, a solution, a suspension, a patch, an ointment and a mask.

28. The method of claim 25, wherein said rapamycin is present in said formulation at a concentration of about 0.01% to about 10% by weight.

29. The method of claim 28, wherein said rapamycin is present in said formulation at a concentration of about 1% to about 8% by weight.

30. The method of claim 25, wherein said formulation is administered once a day.

31. The method of claim 25, wherein said formulation is administered twice a day.

32. The method of claim 25, further comprising administering one or more tetracyclines orally to said patient.

33. The method of claim 25, further comprising administering metronidazole or azelic acid topically to said patient.

34. The method of claim 25, further comprising administering vascular laser therapy to said patient.

35. A method for treating rosacea exhibiting only an erythematotelangiectatic subtype comprising administering topically to a patient a formulation comprising about 0.01% to about 10% rapamycin by weight, wherein said rapamycin is the only active agent in said formulation, thereby reducing erythematotelangiectasia of said rosacea.

36. The method of claim 35, wherein said formulation comprises about 1% to about 8% rapamycin by weight.

37. The method of claim 35, wherein said formulation is administered to said patient's face.

38. The method of claim 35, wherein said formulation is selected from the group consisting of a cream, a lotion, a spray, a foam, a gel, a solution, a suspension, a patch an ointment and a mask.

39. The method of claim 35, wherein said formulation is administered once a day.

40. The method of claim 35, wherein said formulation is administered twice a day.

41. The method of claim 35, further comprising administering one or more tetracyclines orally to said patient.

42. The method of claim 35, further comprising administering metronidazole or azelic acid topically to said patient.

43. The method of claim 35, further comprising administering vascular laser therapy to said patient.

44. A method for preventing a skin condition that exhibits telangiectasia comprising administering to a patient a formulation comprising a therapeutically effective amount of an mTOR inhibitor, wherein said mTOR inhibitor is the only active agent in said formulation, thereby reducing formation of said telangiectasia.

45. The method of claim 44, wherein said mTOR inhibitor is rapamycin.

46. The method of claim 44, wherein said skin condition is selected from the group consisting of rosacea, keratosis pilaris, photodamage, angiofibroma, port wine stain, cutis marmaorata telangiectatica congenita and hemangioma.

47. The method of claim 44, wherein said formulation is administered topically.

48. The method of claim 47, wherein said formulation is administered to said patient's face.

49. The method of claim 47, wherein said formulation is selected from the group consisting of a cream, a lotion, a spray, a foam, a gel, a solution, a suspension, a patch, an ointment and a mask.

50. The method of claim 44, wherein said mTOR inhibitor is present in said formulation at a concentration of about 0.01% to about 10% by weight.

51. The method of claim 50, wherein said mTOR inhibitor is present in said formulation at a concentration of about 1% to about 8% by weight.

52. The method of claim 44, wherein said formulation is administered once a day.

53. The method of claim 44, wherein said formulation is administered twice a day.

54. The method of claim 44, further comprising administering one or more tetracyclines orally to said patient.

55. The method of claim 44, further comprising administering metronidazole or azelic acid topically to said patient.

56. The method of claim 44, further comprising administering vascular laser therapy to said patient.

57. A method for preventing rosacea that exhibits an erythematotelangiectatic subtype comprising administering topically to a patient a formulation comprising a therapeutically effective amount of rapamycin, wherein said rapamycin is the only active agent in said formulation, thereby reducing formation of erythematotelangiectasia of said rosacea.

58. The method of claim 57, wherein said formulation is administered to said patient's face.

59. The method of claim 58 wherein said formulation is selected from the group consisting of a cream, a lotion, a spray, a foam, a gel, a solution, a suspension, a patch an ointment and a mask.

60. The method of claim 57, wherein said rapamycin is present in said formulation at a concentration of about 0.01% to about 10% by weight.

61. The method of claim 60, wherein said rapamycin is present in said formulation at a concentration of about 1% to about 8% by weight.

62. The method of claim 57, wherein said rosacea exhibits only an erythematotelangiectatic subtype.

63. The method of claim 57, wherein said formulation is administered once a day.

64. The method of claim 57, wherein said formulation is administered twice a day.

65. The method of claim 57, further comprising administering one or more tetracyclines orally to said patient.

66. The method of claim 57, further comprising administering metronidazole or azelic acid topically to said patient.

67. The method of claim 57, further comprising administering vascular laser therapy to said patient.

68. A method for preventing rosacea that exhibits an erythematotelangiectatic subtype comprising administering topically to a patient a formulation consisting essentially of a therapeutically effective amount of rapamycin, thereby reducing formation of erythematotelangiectasia of said rosacea.

69. The method of claim 68, wherein said formulation is administered to said patient's face.

70. The method of claim 68, wherein said formulation is selected from the group consisting of a cream, a lotion, a spray, a foam, a gel, a solution, a suspension, a patch an ointment and a mask.

71. The method of claim 68, wherein said rapamycin is present in said formulation at a concentration of about 0.01% to about 10% by weight.

72. The method of claim 71, wherein said rapamycin is present in said formulation at a concentration of about 1% to about 8% by weight.

73. The method of claim 68, wherein said formulation is administered once a day.

74. The method of claim 68, wherein said formulation is administered twice a day.

75. The method of claim 68, further comprising administering one or more tetracyclines orally to said patient.

76. The method of claim 68, further comprising administering metronidazole or azelic acid topically to said patient.

77. The method of claim 68, further comprising administering vascular laser therapy to said patient.

78. A method for preventing rosacea exhibiting only an erythematotelangiectatic subtype comprising administering topically to a patient a formulation comprising about 0.01% to about 10% rapamycin by weight, wherein said rapamycin is the only active agent in said formulation, thereby reducing formation of erythematotelangiectasia of said rosacea.

79. The method of claim 78, wherein said formulation comprises about 1% to about 8% rapamycin by weight.

80. The method of claim 78, wherein said formulation is administered to said patient's face.

81. The method of claim 78, wherein said formulation is selected from the group consisting of a cream, a lotion, a spray, a foam, a gel, a solution, a suspension, a patch, an ointment and a mask.

82. The method of claim 78, wherein said formulation is administered once a day.

83. The method of claim 78, wherein said formulation is administered twice a day.

84. The method of claim 78, further comprising administering one or more tetracyclines orally to said patient.

85. The method of claim 78, further comprising administering metronidazole or azelic acid topically to said patient.

86. The method of claim 78, further comprising administering vascular laser therapy to said patient.
